(19) United States
(12) Patent Application Publication
(10) Pub. No.: US 2026/0325490 A1
(43) Pub. Date: Oct. 22, 2026

(54) ORAL FORMULATIONS OF EDARAVONE AND IMPROVED METHOD OF MANUFACTURING THEREOF

(71) Applicant: BDR PHARMACEUTICALS INTERNATIONAL PRIVATE LIMITED, Mumbai, Maharashtra (IN)

(72) Inventors: Dharmesh Mahendrabhai SHAH, Mumbai (IN); Aravind Manappa BADIGER, Halol, Gujarat (IN); Mukeshkumar Subhashchandra SHARMA, Halol, Gujarat (IN); Madhavkumar Dilipbhai TRIVEDI, Halol, Gujarat (IN); Ronak Prafulchandra DUDHAWALA, Halol, Gujarat (IN); Harsh Kamleshbhai VARIA, Halol, Gujarat (IN); Tejas Nathalal SAVANIYA, Halol, Gujarat (IN)

(21) Appl. No.: 19/104,396

(22) PCT Filed: Aug. 18, 2023
(86) PCT No.: PCT/IN2023/050783

§ 371 Date: Feb. 18, 2025

(30) Foreign Application Priority Data
Aug. 18, 2022 (IN) 202221046887

(51) Int. Cl. A61K 31/4152; A61K 9/00; A61K 9/16

(52) U.S. Cl. CPC A61K 31/4152; A61K 9/0095; A61K 9/1611; A61K 9/1617; A61K 9/1623; A61K 9/1641; A61K 9/1652; A61K 9/1694

(57) ABSTRACT

The present invention relates to a powder for oral suspension dosage form of Edaravone. The invention also relates to provide patient-compliant, economical and technically advanced dosage form over existing dosage form. Moreover, the solubility and stability of the patient compliant Edaravone formulation, prepared as per the present invention, is proven higher when compared to prior art inventions. Furthermore, the present invention also provides a powder for oral suspension composition prepared by an improved process which is relatively simple, easy to commercially manufacture, and functionally reproducible.

### FIELD OF THE INVENTION

The present invention relates to an Edaravone powder for oral suspension. Further, the present invention relates to providing an economical and technically advanced dosage form over the existing dosage forms. Provided is an Edaravone powder for oral suspension for oral administration that has excellent physical property as well as bioavailability. It is expected that burden on ALS patients and care workers can be reduced thereby.

### BACKGROUND OF THE INVENTION

Edaravone (CAS: 89-25-8) is a member of substituted 2-pyrazolin-5-one chemical moiety. Edaravone is chemically known as [3-methyl-1-phenyl-2-pyrazolin-5-one] and is represented structurally as below:

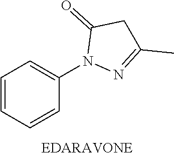

The active ingredient in RADICAVA and RADICAVA ORS is Edaravone, which is a member of the substituted 2-pyrazolin-5-one class. The molecular formula is C₁₀H₁₀N₂O and the molecular weight is 174.20. Edaravone is a white crystalline powder with a melting point of 129.7° C. It is freely soluble in acetic acid, methanol, or ethanol and slightly soluble in water or diethyl ether.

Edaravone is disclosed earlier during 1925 in DE473214. The said German patent discloses a process for the preparation of 1-phenyl-3-methylpyrazolone by the oxidation of 1-phenyl-3-methylpyrazolidone in aqueous hydrochloric acid solution and in the presence of oxygen-transferring metal salts, characterized by the use of oxygen or oxygen-containing metal salts gases as oxidizing agents.

Edaravone is marketed presently under the brand name “RADICAVA®” in United States. This intravenous infusion is indicated for the treatment of amyotrophic lateral sclerosis (ALS). The recommended dosage for Edaravone is 60 mg which is administered as an intravenous infusion over 60 minutes. Prior to US approval, Mitsubishi Tanabe Pharm Corp. (Osaka, Japan) launched Edaravone under the brand name “RADICUT®” in Japan as world's first neurovascular protective drug. RADICUT oral suspension is administered once a day using an oral dosing syringe.

The recommended dose of Edaravone is 60 mg, which should be administered in the form of two consecutive infusion bags containing 60 mg of Edaravone, i.e., 30 mg per each infusion bag over a period of 60 minutes. Each 100 ml infusion bag of RADICAVA® contains Edaravone, L-cysteine hydrochloride hydrate and sodium bisulfate. The pH of the formulation is adjusted with phosphoric acid and sodium hydroxide and the isotonicity is maintained by adding sodium chloride. The dosing regimen of Edaravone consist of 2 cycles. Cycle 1 includes an initial treatment cycle with daily dosing for 14 days; followed by a 14-day drug-free period. Subsequent treatment cycles (cycles 2 to 6) are provided with daily dosing for 10 days out of 14-day periods; followed by 14-day drug-free periods.

Currently, Edaravone is used as an ALS therapeutic agent. However, Edaravone as an ALS therapeutic agent is provided only as an injection or by using an oral dosing syringe. As such, an orally administered drug product is desired that is less burdensome for patients and caregivers and is preferable from a QOL perspective.

However, unlike an injection administered directly into blood, for a drug product for oral administration for which various factors such as absorption from the gastrointestinal tract and a first-pass effect affect bioavailability, obtaining a drug product biologically equivalent to an injection is difficult.

As drug products for oral administration, solid drug products such as tablets and capsules are common. However, when drug products of these forms are taken directly, it is difficult to take the drug products for ALS patients who are expected to have declined swallowing ability. For these patients, oral administration forms such as liquids and oral suspensions are desirable.

U.S. Pat. No. 6,933,310 claims a method for treating motor neuron diseases, amyotrophic lateral sclerosis (ALS), consisting essentially of administering an effective amount of Edaravone or a physiologically acceptable salt thereof to a patient.

CN1241565C discloses a freeze-dried pharmaceutical composition of Edaravone to facilitate storage and transport. As per this Chinese patent, Edaravone powder is suspended in water for injection and alkaline material followed by addition of other scaffolds agent to prepare lyophilized dosage form. However, the said invention is not considered as friendly for long-term treatment from the patient compliance point of view. Therefore, there is a need for a patient compliant invention.

WO2009/067343 discloses an emulsion formulation of wherein alcohol, chelating agent and reducing agents are absent. Further, the emulsifier is chosen from egg-yolk phospholipid or soyabean phospholipid. However, during long-term storage, poor stability of this liquid emulsion dosage form is a hurdle and requires a novel stable dosage form.

WO2012/019381 discloses an oral pharmaceutical composition containing inclusion complex of Edaravone and cyclodextrin. This patent comprises mixing Edaravone with cyclodextrin into water for 1 to 2 h. More contact time with water forces Edaravone to hydrolyze. This invention does improve solubility of Edaravone; however, fails to reduce inherent oxidation of Edaravone. Therefore, there is requirement of a novel invention that yields better solubility with significant stability of Edaravone in the oral dosage form.

WO2017/157350 discloses a lipid-based drug delivery system comprising Edaravone or a pharmaceutically acceptable salt thereof. A solid dispersion comprises Edaravone and a polymeric carrier. However, preparing solid dispersion of Edaravone needs reduction in particle size of API and reduction in particle size yield larger surface area to Edaravone. Thereby, producing higher chance of oxidation to Edaravone due to larger surface area of particles. Therefore, this invention also fails to minimize oxidative effect on Edaravone.

WO2018/133957 claims a solid water-dispersible pharmaceutical composition comprising of dispersing the pharmaceutical composition into an aqueous liquid to produce an enterally administrable liquid containing at least 0.5 grams of the pharmaceutical composition and at least 0.3 g/ml of Edaravone, followed by enterally administering the liquid to a human patient in an amount providing a dose of 30-300 mg Edaravone. The said pharmaceutical composition comprising 2-50 wt. % of Edaravone and 3-50 wt. % of water-soluble alkalizing agent.

WO2018/134243 discloses a liquid pharmaceutical composition which is a monophasic aqueous solution of non-complexed Edaravone that comprise of at least 75 wt. % water and 0.2-9 mg/ml of Edaravone, wherein the treatment comprises oral or gastric administration of 10-250 ml of the liquid pharmaceutical composition to provide 30-300 mg Edaravone.

Toshiaki Sato et. al. (Pharmacology 2010 (85); p. 88-94) describes pharmacokinetics of Edaravone using Edaravone/hydroxypropyl-β-cyclodextrin complex solution, including L-cysteine (L-Cys) and sodium hydrogen sulfite (SHS). This study suggested that L-Cys and SHS were useful for the oral mucosal and rectal administration of Edaravone.

Parikh et al. (International Journal of Pharmaceutics, 2016 (515); p. 490-500) discuss the development of an oral delivery system of Edaravone. The Novel Oral Delivery System (NODS) of Edaravone that is made up of a mixture of Labrasol and an acidic aqueous system that was optimized on the basis of a solubility and stability study. It was found that the oral bioavailability of the NODS delivery system was 5.7 times higher than that of an Edaravone suspension containing 30 mg/mL Edaravone and 0.5% of sodium carboxymethyl cellulose.

Parikh et al. (Drug Delivery 2017; 24 (1); p. 962-978) describe a study that aimed at enabling oral use of Edaravone by developing a lipid-based nanosystem (LNS). The components of LNS including oil, surfactants, and co-surfactants were selected based on their potential to maximize the solubilization in gastrointestinal (GI) fluids, reduce its glucuronidation and improve transmembrane permeability. A liquid LNS (L-LNS) in the form of a micro-emulsion was prepared, comprising Capryol™ PGMC (Oil), Cremophor® RH 40: Labrasol®: TPGS 1000 (1:0.8:0.2) (Surfactant) and Transcutol® P (Co-surfactant). It was found that the oral bioavailability of the L-LNS was almost 11 times higher than that of an Edaravone suspension containing 30 mg/mL Edaravone and 0.5% of sodium carboxymethyl cellulose.

It may be noted that the efficacy of Edaravone has seen an undisputed success since its inception in the therapeutic category of stroke management. Edaravone per se has wide applicability and has potential to be used for more therapeutic indications. The injectable dosage form has been a limiting factor in case of increased use of this product. Importantly, developing an oral dosage form of Edaravone is a challenging process due to its less soluble and less permeable (BCS Class-IV product) nature. Further, an injectable dosage form has inherent difficulty in terms of its usage post hospitalization. Apart from all these, Edaravone is also quite sensitive to hydrolysis and oxidation and thereby making an oral formulation more difficult. The inventors of the present invention during development have noted that it was difficult to stabilize Edaravone. Edaravone has tendency to easily decompose by oxidation in the aqueous solution when compared to the powdered form. Above limitations were identified quite effectively by the inventors of the present invention and hence, a stable-efficacious powder for oral suspension dosage form has been developed to meet the unmet needs in the prior-art.

The present invention discloses the administration of Edaravone in a powder for oral suspension form that does not use sophisticated techniques and is economically affordable compared to parenteral and syringe based oral liquid dosage form. Further, the formulated powder for oral suspension dosage form prepared as per the present invention provides greater stability when compared with other Edaravone formulations. Moreover, the oral route is the preferred one for administration in patients with chronic neurodegenerative diseases. In addition, medical practitioners also prefer powder for oral suspension dosage form over injectable and syringe based oral liquid dosage forms due to advantages like overcoming hydrolysis and oxidation, ease of administration, increased patient compliance, reduced the need for the hospital stay or assisted/monitored administration of Edaravone.

A powder for oral suspension is a heterogeneous mixture that contains solid particles sufficiently large for sedimentation. The suspended particles may be visible to the naked eye which is usually largen than 1 micrometer and/or 1 nanometer. Generally, the solid part is dispersed through mechanical agitation using suspending agents. The present invention overcomes problems of sedimentation and cake formation.

The present invention incorporates Edaravone with stabilizers which reduce the oxidation of Edaravone. Further, smaller particle size of suspension increases the solubility of Edaravone by providing better dissolution profile. Moreover, the present invention resulted in achieving higher stability of Edaravone as comparing to the other existing dosage form. Moreover, the solubility and stability of the patient compliant Edaravone formulation, prepared as per the present invention, is proven higher when compared to prior art inventions.

Further, the present invention provides a powder for oral suspension prepared by a process which is relatively simple, easy to commercially manufacture, and functionally reproducible. Additionally, a suspension composition of the present invention is also able to incorporate two or more active ingredients.

### Objectives of the Invention

The principal object of the present invention is to provide oral dosage forms comprising Edaravone and one or more pharmaceutically acceptable excipients, where in oral dosage forms includes powder for oral suspension as well as other oral dosage forms.

The prime object of the present invention is to provide improved oral pharmaceutical formulations of Edaravone and pharmaceutically acceptable salt thereof in the form of powder in a unit dose sachet for oral suspension.

Other object of the present invention is to provide the composition of Edaravone an economical and advanced dosage form over existing dosage form.

In yet another general object of the present invention is to provide a problem solution approach over the existing dosage form.

Still another object of the present invention is providing an oral dosage form which is in the form of powder for oral suspension with improved taste, thus having high patient compliance.

Yet another object of the present invention is provided composition, which is used as medicament and process for preparation.

### SUMMARY OF THE INVENTION

Despite of wide research on Edaravone as reported in prior-art publications, powder for oral composition of Edaravone is not accessible commercially around the globe. It has thus been unmet need to develop a patient compliant oral composition for Edaravone with good stability.

Accordingly, the present invention provides an oral composition of Edaravone preferably as powder for oral suspension dosage form with pharmaceutically acceptable excipients and method of preparation thereof.

In one general aspect, a pharmaceutical composition as per the present invention is in the form of powder for oral formulation that when reconstituted in water held in supply cup a proper suspension for oral administration is obtained within 1 minute of shaking and the powder did not stick to the wall and bottom of mixing cap

In yet another aspect, a stable pharmaceutical composition as per the present invention comprises Edaravone and one or more pharmaceutically acceptable excipients wherein the composition is in the form of an oral powder suspension.

In one aspect of the present invention, wherein Edaravone particles contained in the suspension of the present invention are solid particles containing Edaravone, and may be formed of Edaravone alone or may contain other components. Edaravone may be in a crystalline state or an amorphous state.

One more aspect of the present invention, wherein the active ingredient incorporated in the pharmaceutical composition comprises D₉₀ of particle size in the range of 1 to 100 microns, preferably, D₉₀ in the range of 2 to 80 microns.

In yet another aspect of the present invention, wherein the pharmaceutical composition manufactured has particle size ranging from nanometer to micrometer, which results in to good in-vitro dissolution profile.

Another aspect according to the present invention, wherein the formulated product manufactured is oral dosage suspension where in it is a powder for oral suspension, which results in to good in-vitro dissolution profile.

Additional aspect according to the present invention, wherein the formulation manufactured has particle size ranging from nanometer to micrometer, which results in to good bio-absorption.

In yet another aspect of the present invention, wherein the pharmaceutical composition manufactured by number of stages in manufacturing process including granulating, homogenization, sonication, mixing and/or evaporation by rapid mixture granulator and/or spray drying.

Additional aspect according to the present invention is that the formulated product is a stabilized powder in a form of a unit dose sachet for oral suspension.

In yet another aspect of the present invention, wherein pH of the pharmaceutical composition is in the range of 2-6.

One aspect of the present invention may include a pharmaceutical composition comprising about 0.1 mg to 2000 mg of Edaravone with pharmaceutically acceptable excipients.

Yet another aspect of the pharmaceutical composition may include Edaravone as an active ingredient with one or more selected from pharmaceutically acceptable excipients like fillers or diluents, solubility enhancing agents, wetting agents or surfactants, solvents, pH adjusting agents, anti-oxidants, stabilizers, flavoring agents, sweeteners and the like.

Another aspect is a kit comprising one or more-unit dose sachets of Edaravone powder, a syringe and supply cup and optionally, a drinking cup for consumption. The Edaravone powder is dispensed in the supply cap and drinking water is added by way of syringe, mixed and consumed by the patient in the drinking cup.

In another aspect of the present invention, pharmaceutical composition is in the form of powder for oral suspension comprising:
a) dispensing all the materials in separate containers.
b) shifting a material API as Edaravone and with one or more pharmaceutically acceptable diluents as part—A;
c) co-shifting of part—A with one or more pharmaceutically acceptable diluents as part—B;
d) shifting one or more pharmaceutically acceptable pH adjusting agents, anti-oxidants, stabilizers, flavoring agents and sweeteners separately as part—C;
e) adding sufficient quantity of one or more pharmaceutically acceptable solubility enhancing agents and surfactant in the solvent as part—D;
f) granulating part—B with the solution of part—D as part—E;
g) drying part—E and shifting the dried granules as part—F;
h) blending part—C with part—F as part—G;
i) packing powder of part—G in suitable sachets.

The details of one or more embodiments of the invention are set forth in the description below. Other features of the invention will be apparent from the description.

### DETAILED DESCRIPTION OF THE INVENTION

The present invention will now be disclosed by describing certain preferred and optional embodiments, to facilitate various aspects thereof.

Edaravone, chemically known as, 3-methyl-1-phenyl-2-pyrazolin-5-one, is a small lipophilic molecule with molecular weight 174.203 g/mol with a solubility of 25 μg/ml. Edaravone is considered a Bio-pharmaceutics Classification System (BCS) Class IV drug substance due to its poor solubility in water and lower lipid permeability. The mean terminal elimination half-life of Edaravone is 4.5 to 6 h.

References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The term “drug” or “active ingredient” or “active pharmaceutical ingredient (API)” herein refers to Edaravone or a pharmaceutically acceptable salt thereof.

In accordance with the present invention, a pharmaceutical composition of Edaravone comprising of Edaravone as an active ingredient with pharmaceutically acceptable excipients.

The term “Edaravone” as used herein according to the present invention includes, Edaravone in the form of free base, a pharmaceutically acceptable salt thereof, amorphous, crystalline, any isomer, derivative, hydrate, solvate or prodrug or a combination thereof.

The term “pharmaceutical composition” includes oral dosage forms, such as but not limited to, tablets, soft gelatin capsule, capsules (filled with powders, powders for reconstitution, pellets, beads, mini-tablets, pills, micro-pellets, small tablet units, multiple unit pellet system (MUPS), disintegrating tablets, dispersible tablets, granules, sprinkles microspheres and multi-particulate), sachets (filled with powders, pellets, beads, mini-tablets, pills, micro-pellets, small tablet units, MUPS, disintegrating tablets, dispersible tablets, granules, sprinkles microspheres and multi-particulates) and sprinkles.

The term “oral dosage forms” as may include one or more of forms Syrup, oral solution, oral suspension, oral drop, oral emulsion, Mixture, Linctus, Elixir and like.

The term “pharmaceutically acceptable excipients” as used herein, refers to excipients those are routinely used in pharmaceutical compositions. The pharmaceutically acceptable excipients may comprise of fillers or diluents, solubility enhancing agents, wetting agents or surfactants, solvents, pH adjusting agents, anti-oxidants, stabilizers, flavoring agents, sweeteners and combinations thereof.

Suitable fillers or diluents may include one or more of sugars such as lactose, sucrose glucose, fructose, dextrose, galactose, starch; cellulose derivatives such as microcrystalline cellulose; carbonates like calcium carbonate; sugar alcohols such as mannitol, sorbitol, erythritol; magnesium carbonate, calcium phosphates kaolin, magnesium oxide, magnesium hydroxide; and cellulose derivative such as Avicel CL-611 (Microcrystalline cellulose and Carboxymethylcellulose sodium) and the like.

Suitable solubility enhancing agents may include one or more from the group comprising surfactants such as (1) non-ionic e.g., polyoxyethylene sorbitan fatty acid esters, sorbitan esters, polyoxyethylene ethers, and the like. (2) anionic e.g., sodium lauryl sulfate, sodium laurate, dialkyl sodium sulfosuccinates, particularly bis-(2-ethylhexyl) sodium sulfosuccinate, sodium stearate, potassium stearate, and sodium oleate, and the like. (3) cationic e.g., benzalkonium chloride and bis-2-hydroxyethyl oleyl amine, and the like. (4) zwitterionic/amphoteric surfactants; fatty alcohols such as lauryl, cetyl, and stearyl alcohols; glyceryl esters such as the naturally occurring mono-, di-, and tri-glycerides; fatty acid esters of fatty alcohols and other alcohols such as propylene glycol, polyethylene glycol; sucrose; polymers e.g., poloxamers, polyvinylpyrrolidones, glycerides e.g., triacetin, glyceryl monocaprylate, glyceryl monooleate, glyceryl monostearate; diethylene glycol monoethyl ether, Labrafil M 1944 CS; and the like.

Suitable wetting agents or surfactants may include but are not limited to one or more from the group comprising sodium lauryl sulphate, cetrimide, polyethylene glycols, polyoxyethylene-polyoxypropylene block copolymers such as poloxamers, pluronic F68, polyglycerin fatty acid esters such as decaglyceryl monolaurate and decaglyceryl monomyristate, sorbitan fatty acid esters such as sorbitan monostearate, polyoxyethylene sorbitan fatty acid esters such as polyoxyethylene sorbitan monooleate, polyethylene glycol fatty acid esters such as polyoxyethylene monostearate, polyoxyethylene alkyl ethers such as polyoxyethylene lauryl ether, polyoxyethylene castor oil, sepitrap 80, polysorbate 80 (Tween 80, polyoxyethylene sorbitan monooleate), Labrafil M 1944 CS and the like.

polyethylene glycols, polyoxyethylene-polyoxypropylene block copolymers such as poloxamers, pluronic F68, polyglycerin fatty acid esters such as decaglyceryl monolaurate and decaglyceryl monomyristate, sorbitan fatty acid esters such as sorbitan monostearate, polyoxyethylene sorbitan fatty acid esters such as polyoxyethylene sorbitan monooleate, polyethylene glycol fatty acid esters such as polyoxyethylene monostearate, polyoxyethylene alkyl ethers such as polyoxyethylene lauryl ether, polyoxyethylene castor oil, sepitrap 80, polysorbate 80 (Tween 80, polyoxyethylene sorbitan monooleate), PEG 6000, PEG 400, and the like.

Solvents play key roles in designing drug delivery systems (DDSs). Specifically, organic solvents are commonly used in the pharmaceutical industry as reaction media. Solvents are uniquely able to dissolve drugs safely and effectively into these medicinal formulations. Further, Solvent Suitable organic solvents for preparing binding solution may include one or more of water, organic solvents such as ethanol, isopropyl alcohol (IPA), acetone, propylene glycol, Glycerin and the like.

pH adjusting agents may include one or more from Acetic Acid, Aminoethyl Ethanolamine, Barium Carbonate, Caustic Potash, Caustic Soda, Citric Acid Anhydrous, Citric Acid (Industrial Grade), Citric Acid, Aqueous Solution (Food Grade), Ammonium Bicarbonate, Calcium Acid Pyrophosphate, Gluconic Acid, Lactic Acid, Magnesium Carbonate, Sodium Bisulphite, and the like.

Suitable anti-oxidants may include one or more from citric acid, butylated hydroxytoluene, butylated hydroxy anisole, Sodium Bisulphite, ascorbic acid, magnesium bisulfite, sodium metabisulfite, tocopherol, ubiquinol, β-carotenes, uric acid, lipoic acid, propyl gallate, thiourea, glutathione and the like.

Suitable stabilizers may include one or more from citric acid, butylated hydroxytoluene, butylated hydroxy anisole, ascorbic acid, L-cysteine HCl, magnesium bisulfite, sodium metabisulfite, tocopherol, ubiquinol, β-carotenes, uric acid, lipoic acid, propyl gallate, thiourea, glutathione and the like.

Suitable flavoring agents may include one or more from the group consisting of peppermint, grapefruit, orange, lime, lemon, mandarin, pineapple, strawberry, raspberry, mango, passion fruit, kiwi, apple, pear, peach, apricot, cherry, grape, banana, cranberry, blueberry, black currant, red currant, gooseberry, lingon berries, cumin, thyme, basil, camille, valerian, fennel, parsley, chamomile, tarragon, lavender, dill, bargamot, salvia, aloe vera balsam, spearmint, piperine, eucalyptus, and the like.

Sweeteners are employed in oral suspension pharmaceutical dosage forms intended for oral administration specifically to increase the palatability of the therapeutic agent. Further, suitable sweeteners may include one or more of Neohesperidine DC/Neotame, Sucralose, Aspartame, Neotame, Saccharin Sodium, Sucrose, Glycyrrhiza glabra, acesulfame potassium, sorbitol, mannitol, xylitol, high fructose corn syrup,

Suitable suspending or thickening agents may include one or more from sodium alginate, methylcellulose, hydroxyethyl cellulose, hydroxypropyl methylcellulose, carboxymethyl cellulose, sodium carboxymethyl cellulose, microcrystalline cellulose, xanthan gum, acacia, tragacanth, bentonite, carbomer, carrageen, gelatin and the like.

One embodiment of the present invention may include a pharmaceutical composition comprising about 0.1 mg to 2000 mg of Edaravone with pharmaceutically acceptable excipients.

Another embodiment of the present invention includes a pharmaceutical composition comprising Edaravone with pharmaceutically acceptable excipients with pH adjusting agents.

In one embodiment of the present invention, wherein the active ingredient incorporated in the pharmaceutical composition comprises D₉₀ of particle size in the range of 1 to 100, preferably, D₉₀ in the range of 2 to 80 microns.

In one of the embodiments, the pharmaceutical composition manufactured as per the present invention is powder for oral suspension.

In yet another embodiment of the present invention, wherein the pharmaceutical composition manufactured is oral dosages suspension, which results in to good in-vitro dissolution profile.

Another embodiment according to the present invention, wherein the formulated product manufactured is oral dosage suspension where in it is a powder for oral suspension, which results in to good in-vitro dissolution profile.

Additional embodiment according to the present invention, wherein the formulation manufactured is powder for oral suspension, which results in to good bio-absorption.

In yet another embodiment of the present invention, wherein the pharmaceutical composition manufactured by number of stages in manufacturing process including homogenization, granulation, sonication, mixing and/or evaporation by rapid mixture granulator and/or spray drying.

Additional embodiment according to the present invention is that the formulated product is a very stabilized powder for oral suspension.

In yet another embodiment of the present invention, wherein pH of the pharmaceutical composition is in the range of 2-6.

In another embodiment, the process comprises a step for providing Edaravone, wherein the Edaravone is contained part—A that may be prepared by mixing Edaravone with one or more pharmaceutically acceptable diluents.

Another embodiment, the process comprises a step co-sifting of part—A with one or more pharmaceutically acceptable diluents and consider as part—B.

One more embodiment, the process comprises a step shifting one or more pharmaceutically acceptable pH adjusting agents, anti-oxidants, stabilizers, flavoring agents and sweeteners separately and consider as part—C.

Yet another embodiment, the process comprises a step adding sufficient quantity of one or more pharmaceutically acceptable solubility enhancing agents and surfactant in the solvent and consider as part—D.

In one embodiment, the process comprises a step granulating part—B with the solution of part—D and consider as part—E.

One more embodiment, the process comprises a step drying part—E and shifting the dried granules and consider as part—F.

In yet another embodiment, the process comprises a step blending part—C with part—F and consider as part—G.

One more embodiment, the process comprises a step packing powder of part—G in suitable sachets.

Another embodiment is a kit comprising one or more-unit dose sachets of Edaravone powder, a syringe and supply cup and optionally, a drinking cup for consumption. The Edaravone powder is dispensed in the supply cap and drinking water is added by way of syringe, mixed and consumed by the patient in the drinking cup.

In another embodiment, the process comprises a step for providing above-mentioned formulation, wherein the formulation is a powder for oral suspension that may be prepared by the steps comprising:
a) dispensing all the materials in separate containers;
b) shifting a material API as Edaravone and with one or more pharmaceutically acceptable diluents as part—A;
c) co-shifting of part—A with one or more pharmaceutically acceptable diluents as part—B;
d) shifting one or more pharmaceutically acceptable pH adjusting agents, anti-oxidants, stabilizers, flavoring agents and sweeteners separately as part—C;
e) adding sufficient quantity of one or more pharmaceutically acceptable solubility enhancing agents and surfactant in the solvent as part—D;
f) granulating part—B with the solution of part—D as part—E;
g) drying part—E and shifting the dried granules as part—F;
h) blending part—C with part—F as part—G;
i) packing powder of part—G in suitable sachets.

The invention will be further described with respect to the following examples; however, the scope of the invention is not limited thereby. All percentages stated in this specification are by weight, unless otherwise specified. While the present invention has been described in terms of its specific embodiments, certain modifications and equivalents will be apparent to those skilled in the art and are intended to be included within the scope of the present invention.

### EXAMPLE 1

TABLE 1
Edaravone Oral Suspension, 105 mg/sachet (1.5 gm)

| Sr. No. | Material Name | % w/w | mg/sachet |
|---|---|---|---|
| 1 | Edaravone | 7.00 | 105.00 |
| 2 | Sorbitol Powder | 83.96 | 1241.85 |
| 3 | Xanthan Gum | 1.00 | 15 |
| 4 | Polyethylene Glycol 400 | 0.50 | 25.05 |
| 5 | Polysorbate 80 | 2.33 | 34.95 |
| 6 | Labrafil M | 3.33 | 49.95 |
| 7 | Isopropyl Alcohol (IPA) | — | — |
| 8 | Citric Acid | 0.17 | 2.55 |
| 9 | Sodium Bisulphite | 0.30 | 4.50 |
| 10 | L-Cysteine HCl | 0.30 | 4.50 |
| 11 | Strawberry Flavor | 1.00 | 15.00 |
| 12 | Sucralose | 0.11 | 1.65 |

| | | |
|---|---|---|
| Total | 100.00 | 1500.00 |

### Manufacturing Procedure:

A. Dispense all the materials in separate containers as per required quantity.
B. Sift Sorbitol powder through 60 #sieve.
C. Co-Sift Edaravone and Sorbitol through 60 #sieve.
D. Co-Sift above mixture of Step C, with Xanthan Gum.
E. Sift Citric Acid Anhydrous, Sodium Bisulphite, L-Cysteine HCl, Strawberry Flavor and Sucralose separately from 60 #Sieve and Store it aside.
F. Take sufficient quantity of Isopropyl Alcohol into S.S. Vessel. Start stirring with slow speed. Add Polysorbate 80, Labrafil M 1944 CS and Polyethylene Glycol 400 into it under stirrer. Perform stirring to form clear solution.
G. Transfer Material of Step D into the Rapid Mixer Granulator and carry out dry mixing.
H. Carry out granulation of Step G with the Solution of Step F.
I. Transfer the wet granules of Step H into Fluid Bed Dryer. Set parameters to achieve required Product Temperature and continue the process till desired drying achieved.
J. Sift the granules of Step I, through 60 #.
K. Transfer the sifted powder of Step J into the blender.
L. Transfer the sifted materials of Step E into the blender.
M. Carry out blending for 10 minutes.
N. Final Blend of Step M, to be packed in double lined polyethylene bag, with silica bag in between polyethylene bags and same polyethylene bag to be kept in Aluminum Bag, with proper sealing.
O. Store it in air tight container.
P. Final packed material should be kept in proper storage condition.
Q. Carry out filling and sealing of sachets.
R. Store the sachets in proper condition.

### EXAMPLE 2

TABLE 2
Edaravone Oral Suspension, 105 mg/sachet (1.5 gm)

| Sr. No. | Material Name | % w/w | mg/sachet |
|---|---|---|---|
| 1 | Edaravone | 7.00 | 105.00 |
| 2 | Sorbitol Powder | 83.96 | 1241.85 |
| 3 | Sodium Carboxy Methyl Cellulose | 1.00 | 15 |
| 4 | Polyethylene Glycol 400 | 0.50 | 25.05 |
| 5 | Polysorbate 80 | 2.33 | 34.95 |
| 6 | Labrafil M | 3.33 | 49.95 |
| 7 | Isopropyl Alcohol (IPA) | — | — |
| 8 | Citric Acid | 0.17 | 2.55 |
| 9 | Sodium Bisulphite | 0.30 | 4.50 |
| 10 | L-Cysteine HCl | 0.30 | 4.50 |
| 11 | Strawberry Flavor | 1.00 | 15.00 |
| 12 | Sucralose | 0.11 | 1.65 |

| | | |
|---|---|---|
| Total | 100.00 | 1500.00 |

### Manufacturing Procedure:

A. Dispense all the materials in separate containers as per required quantity.
B. Sift Sorbitol powder through 60 #sieve.
C. Co-Sift Edaravone and Sorbitol through 60 #sieve.
D. Co-Sift above mixture of Step C, with Sodium Carboxy Methyl Cellulose.
E. Sift Citric Acid Anhydrous, Sodium Bisulphite, L-Cysteine HCl, Strawberry Flavor and Sucralose separately from 60 #Sieve and Store it aside.
F. Take sufficient quantity of Isopropyl Alcohol into S.S. Vessel. Start stirring with slow speed. Add Polysorbate 80, Labrafil M 1944 CS and Polyethylene Glycol 400 into it under stirrer. Perform stirring to form clear solution.
G. Transfer Material of Step D into the Rapid Mixer Granulator and carry out dry mixing.
H. Carry out granulation of Step G with the Solution of Step F.
I. Transfer the wet granules of Step H into Fluid Bed Dryer. Set parameters to achieve required Product Temperature and continue the process till desired drying achieved.
J. Sift the granules of Step I, through 60 #.
K. Transfer the sifted powder of Step J into the blender.
L. Transfer the sifted materials of Step E into the blender.
M. Carry out blending for 10 minutes.
N. Final Blend of Step M, to be packed in double lined polyethylene bag, with silica bag in between polyethylene bags and same polyethylene bag to be kept in Aluminum Bag, with proper sealing.
O. Store it in air tight container.
P. Final packed material should be kept in proper storage condition.
Q. Carry out filling and sealing of sachets.
R. Store the sachets in proper condition.

### EXAMPLE3

Edaravone Oral Suspension, 105 mg/sachet (1.5 gm)

| Sr. No. | Material Name | % w/w | mg/sachet |
|---|---|---|---|
| 1 | Edaravone | 7.00 | 105.00 |
| 2 | Sorbitol Powder | 74.96 | 1124.40 |
| 3 | Avicel CL-611 | 10.00 | 150.00 |
| | (Microcrystalline cellulose | | |
| | and Carboxymethylcellulose | | |
| | sodium) | | |
| 4 | Polyethylene Glycol 400 | 0.50 | 7.50 |
| 5 | Polysorbate 80 | 2.33 | 34.95 |
| 6 | Labrafil M | 3.33 | 49.95 |
| 7 | Isopropyl Alcohol (IPA) | — | — |
| 8 | Citric Acid | 0.17 | 2.55 |
| 9 | Sodium Bisulphite | 0.30 | 4.50 |
| 10 | L-Cysteine HCl | 0.30 | 4.50 |
| 11 | Strawberry Flavor | 1.00 | 15.00 |
| 12 | Sucralose | 0.11 | 1.65 |

| | | |
|---|---|---|
| Total | 100.00 | 1500.00 |

### Manufacturing Procedure:

A. Dispense all the materials in separate containers as per required quantity.
B. Sift Sorbitol powder through 60 #sieve.
C. Co-Sift Edaravone and Sorbitol through 60 #sieve.
D. Co-Sift above mixture of Step C, with Xanthan Gum.
E. Sift Citric Acid Anhydrous, Sodium Bisulphite, L-Cysteine HCl, Strawberry Flavor and Sucralose separately from 60 #Sieve and Store it aside.
F. Take sufficient quantity of Isopropyl Alcohol into S.S. Vessel. Start stirring with slow speed. Add Polysorbate 80, Labrafil M 1944 CS and PEG 400 into it under stirrer. Perform stirring to form clear solution.
G. Transfer Material of Step D into the Rapid Mixer Granulator and carry out dry mixing.
H. Carry out granulation of Step G with the Solution of Step F.
I. Transfer the wet granules of Step H into Fluid Bed Dryer. Set parameters to achieve required Product Temperature and continue the process till desired drying achieved.
J. Sift the granules of Step I, through 60 #.
K. Transfer the sifted powder of Step J into the blender.
L. Transfer the sifted materials of Step E into the blender.
M. Carry out blending for 10 minutes.
N. Final Blend of Step M, to be packed in double lined polyethylene bag, with silica bag in between polyethylene bags and same polyethylene bag to be kept in Aluminum Bag, with proper sealing.
O. Store it in air tight container.
P. Final packed material should be kept in proper storage condition.
Q. Carry out filling and sealing of sachets.
R. Store the sachets in proper condition.

### Observation:

The major challenges during the development were to achieve the desired reconstitution time and non-stickiness of the powder, since the formulation is in powder form and is administered after reconstitution in the suspension form. If there is any loss of powder, due to sticking on the surface of the sachet, mixing cap or the supply container, it may cause 5 the improper dosing of the drug, thus failing to achieve proper therapeutic dose.

During reconstitution of powder to suspension, the powder did not stick to the wall and bottom of mixing cap and proper suspension is obtained. Desirable Reconstitution time observed, i.e. within 1 Minute.

### Assay and Dissolution of Edaravone Oral Suspension:

Assay and dissolution profile studies were incorporated at various time periods. The dissolution profile studies were observed in media as defined by USFDA, which is having 30 rpm in 900 mL of 0.1 N Hydrochloric acid. For this stability study, the oral suspension composition prepared according to Example-3 were observed.

Dissolution of Edaravone Oral Suspension, 105 mg/sachet (1.5 gm)

| Dissolution Media | 0.1N Hydrochloric acid | |
|---|---|---|
| Dissolution Media Volume | 900 | ml |
| USP Apparatus | Paddle | |
| RPM | 30 | RPM |

| Time point (min) | % Drug Release of Edaravone |
|---|---|
| 5 | 83.4% |
| 10 | 89.4% |
| 15 | 92.9% |
| 20 | 95.2% |
| 30 | 98.3% |
| 45 | 100.0% |
| Assay (%) | 99.23% |

The invention is storage stable for prolonged period of time. The composition is stable at 30° C./75% RH and 40° C./75% RH and there is no change in colour and/or taste.

The invention described herein comprises in various objects as mentioned above and their description in relation to characteristics, compositions and process adopted. While these aspects are emphasised in the invention, any variations of the invention described above are not to be regarded as departure from the spirit and scope of the invention as described.

The above-mentioned examples are provided for illustrative purpose only and these examples are in no way limitative on the present invention.

1. A pharmaceutical composition in the form of reconstitutable powder for oral suspension comprising edaravone or a pharmaceutically acceptable salt thereof and one or more pharmaceutically acceptable excipients.

2. The composition as claimed in claim 1, comprising edaravone or a pharmaceutically acceptable salt in the form of solid particles having a particle size distribution wherein D90 is in the range of 1 to 100 microns.

3. The composition as claimed in claim 1, comprising about 0.005% to 95% w/v of edaravone with one or more pharmaceutically acceptable excipients.

4. The composition as claimed in claim 1, wherein the one or more pharmaceutically acceptable excipients are selected from like one or more of fillers or diluents, solubility enhancing agents, wetting agents or surfactants, solvents, pH adjusting agents, anti-oxidants, stabilizers, flavoring agents, and sweeteners.

5. The composition as claimed in claim 1, comprising
(a) 0.005% to 95% w/v of edaravone;
(b) 2% to 99% w/v of fillers or diluents;
(c) 0.05% to 65% w/v of solubility enhancing agents;
(d) 0.05% to 55% w/v of wetting agents or surfactants;
(e) 0.0025% to 25% w/v of pH adjusting agents;
(f) 0.001% to 15% w/v of anti-oxidants;
(g) 0.001% to 15% w/v of stabilizers;
(h) 0.005% to 50% w/v of flavoring agents; and
(i) 0.001% to 10% w/v of sweeteners.

6. The composition as claimed in claim 1, comprising:
(a) edaravone in a concentration from about 0.005% by weight to about 95% by weight of the composition;
(b) a fillers or diluent component in a concentration from about 2% by weight to about 99% by weight of the composition, the fillers or diluent component comprising one or more pharmaceutically acceptable fillers or diluent components are selected from the group consisting of one or more of sugars such as lactose, sucrose glucose, fructose, dextrose, galactose, starch; cellulose derivatives such as microcrystalline cellulose; carbonates like calcium carbonate; sugar alcohols such as mannitol, sorbitol, erythritol; magnesium carbonate, calcium phosphates kaolin, magnesium oxide, magnesium hydroxide; and cellulose derivative such as Avicel CL-611 (microcrystalline cellulose and carboxymethylcellulose sodium);
(c) a solubility enhancing agent component in a concentration from about 0.05% by weight to about 65% by weight to about by weight of the composition, the solubility enhancing agent component comprising one or more pharmaceutically acceptable solubility enhancing agent components are selected from the group consisting of one or more of (1) non-ionic e.g., polyoxyethylene sorbitan fatty acid esters, sorbitan esters, polyoxyethylene ethers, and the like; (2) anionic e.g., sodium lauryl sulfate, sodium laurate, dialkyl sodium sulfosuccinates, particularly bis-(2-ethylhexyl) sodium sulfosuccinate, sodium stearate, potassium stearate, and sodium oleate, and the like; (3) cationic e.g., benzalkonium chloride and bis-2-hydroxyethyl oleyl amine, and the like; (4) zwitterionic/amphoteric surfactants; fatty alcohols such as lauryl, cetyl, and stearyl alcohols; glyceryl esters such as the naturally occurring mono-, di-, and tri-glycerides; fatty acid esters of fatty alcohols and other alcohols such as propylene glycol, polyethylene glycol; sucrose; polymers e.g., poloxamers, polyvinylpyrrolidones, glycerides e.g., triacetin, glyceryl monocaprylate, glyceryl monooleate, glyceryl monostearate; diethylene glycol monoethyl ether, Labrafil M 1944 CS;
(d) a wetting agents or surfactant component in a concentration from about 0.05% by weight to about 55% by weight of the composition, the wetting agents or surfactant component comprising at least one pharmaceutically acceptable wetting agents or surfactant selected from the group consisting of one or more of sodium lauryl sulphate, cetrimide, polyethylene glycols, polyoxyethylene-polyoxypropylene block copolymers such as poloxamers, pluronic F68, polyglycerin fatty acid esters such as decaglyceryl monolaurate and decaglyceryl monomyristate, sorbitan fatty acid esters such as sorbitan monostearate, polyoxyethylene sorbitan fatty acid esters such as polyoxyethylene sorbitan monooleate, polyethylene glycol fatty acid esters such as polyoxyethylene monostearate, and polyoxyethylene alkyl ethers such as polyoxyethylene lauryl ether, polyoxyethylene castor oil, sepitrap 80, polysorbate 80 (Tween 80, polyoxyethylene sorbitan monooleate), Labrafil M 1944 CS;
(e) a pH adjusting agent component in a concentration from about 0.0025% by weight to about 25% by weight of the composition, the pH adjusting agent component comprising at least one pharmaceutically acceptable pH adjusting agent selected from the group consisting of one or more of acetic acid, aminoethyl ethanolamine, barium carbonate, caustic potash, caustic soda, citric acid anhydrous, citric acid (industrial drade), citric acid, aqueous solution (food grade), ammonium bicarbonate, calcium acid pyrophosphate, gluconic acid, lactic acid, magnesium carbonate, and sodium bisulphite;
(f) an anti-oxidant component in a concentration from about 0.001% by weight to about 15% by weight of the composition, the anti-oxidant component comprising at least one pharmaceutically acceptable anti-oxidant selected from the group consisting of one or more of citric acid, butylated hydroxytoluene, butylated hydroxy anisole, sodium bisulphite, ascorbic acid, magnesium bisulfite, sodium metabisulfite, tocopherol, ubiquinol, β-carotenes, uric acid, lipoic acid, propyl gallate, thiourea, and glutathione;
(g) a stabilizer component in a concentration from about 0.001% by weight to about 15% by weight of the composition, the stabilizer component comprising at least one pharmaceutically acceptable stabilizer selected from the group consisting of one or more of citric acid, butylated hydroxytoluene, butylated hydroxy anisole, ascorbic acid, L-cysteine HCl, magnesium bisulfite, sodium metabisulfite, tocopherol, ubiquinol, β-carotenes, uric acid, lipoic acid, propyl gallate, thiourea, and glutathione;
(h) a flavoring agent component in a concentration from about 0.005% by weight to about 50% by weight of the composition, the flavoring agent component comprising at least one pharmaceutically acceptable flavoring agent; and
(i) a sweetener component in a concentration from about 0.001% by weight to about 10% by weight of the composition, the sweetener component comprising at least one pharmaceutically acceptable sweetener.

7. The composition as claimed in claim 1, comprising the pharmaceutical composition manufactured by the following steps:
(a) dispensing all the materials in separate containers;
(b) shifting a material active pharmaceutical ingredient as edaravone and with one or more pharmaceutically acceptable diluents as part—A;
(c) co-shifting of part—A with one or more pharmaceutically acceptable diluents as part—B;
(d) shifting one or more pharmaceutically acceptable pH adjusting agents, anti-oxidants, stabilizers, flavoring agents and sweeteners separately as part—C;
(e) adding sufficient quantity of one or more pharmaceutically acceptable solubility enhancing agents and surfactant in the solvent as part—D;
(f) granulating part—B with the solution of part—D as part—E;
(g) drying part—E and shifting the dried granules as part—F;
(h) blending part—C with part—F as part—G; and
(i) packing powder of part—G in suitable sachets.

8. The composition as claimed in claim 1, wherein the pH of the pharmaceutical composition is in the range of 2-6.

9. The composition as claimed in claim 1, in the form of an oral suspension reconstituted in water from a unit dose sachet.

10. A kit for administering a pharmaceutical active, the kit comprising:
(a) one or more unit dose sachets of edaravone powder;
(b) a syringe;
(c) a supply cup; and
(d) optionally, a drinking cup for consumption.
